(19) United States
(12) Patent Application Publication
(10) Pub. No.: US 2002/0052413 A1
(43) Pub. Date: May 2, 2002

(54) Treatment of ocular complications

(76) Inventors: Christine Percicot, Strasbourg (FR); George N. Lambrou, Oberdorf (FR); Leopold Schmetterer, Wien (AT)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/906,657

(22) Filed: Jul. 17, 2001

(30) Foreign Application Priority Data
Jul. 19, 2000 (DE) 00115561.3

(51) Int. Cl. A61K 31/557; A61K 31/215

(52) U.S. Cl. 514/559

(57) ABSTRACT

The present invention relates to a method to treat certain ocular complications, comprising the administration of an effective amount of a FAD to a patient in need of such treatment.

The present invention relates to the use of fatty acid derivatives (FADs) and in particular of 13,14-diyhdro-15-keto-prostaglandins in the manufacture of a medicament for the treatment of ocular complications selected from the group of vasospastic glaucoma, vasospastic syndrome, vascular dysregulation syndrome, and signs of vascular dysregulation, especially in patients presenting risk factors for glaucoma, such as but not exclusively high intraocular pressure, family history of glaucoma, glaucoma in the contralateral eye and high myopia.

The present invention also relates to a method to treat certain ocular complications, comprising the administration of an effective amount of a FAD to a patient in need of such treatment.

It has surprisingly been found that certain FADs, preferably 13,14-diyhdro-15-keto-prostaglandins extremely improve ocular complications in patients with elevated levels of endothelin-1(ET-1). Said FADs are therefore in particular suitable for treating inter alia vasospastic ocular disorders. It has been found that the FADs of the present invention can preferably be administered topically to the eye, e.g. as an eye drop. While it is at present not entirely clear, which mechanisms may be responsible for the beneficial efficacy of the addressed prostaglandins, said ocular complications selected from vasospastic glaucoma, vasospastic syndrome, vascular dysregulation syndrome, and signs of vascular dysregulation are highly beneficially influenced, especially in patients presenting risk factors for glaucoma, such as but not exclusively high intraocular pressure, family history of glaucoma, glaucoma in the contralateral eye and high myopia. The FADs of the present invention seem not to exhibit an affinity (or only a very weak) to the ET-receptor. Nevertheless, said FADs appear to antagonize the physiological effect of ET-1 in particular on ocular blood vessels.

Prostaglandins (hereinafter referred to as PGs) is the name given to the group of preferred FADs which show various physiological activities and which are contained in human and animal tissues and organs. PGs essentially contain the prostanoic acid C-20 skeleton of the following formula:

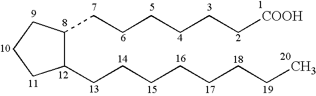

Some synthetic products may also contain the above skeleton with some modification. PGs are classified into several types according to their five-membered ring, for example:

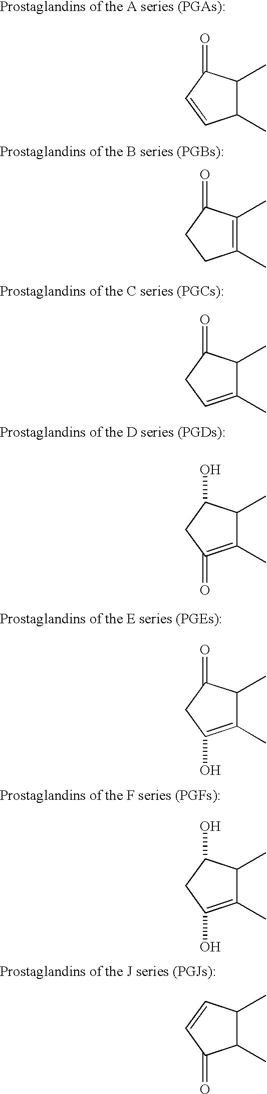

and the like. Further, they are classified into PG₁s containing 5,6-single bond:

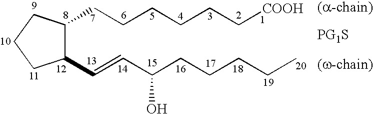

PG₂s containing 5,6-double bond:

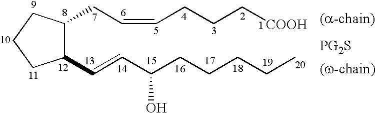

and PG₃s containing 5,6-and 17,18-double bonds:

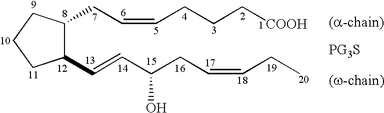

PGs are known to have various pharmacological and physiological activities, for example, vasodilatation, induction of inflammation, platelet aggregation, contraction of uterine muscle, enteron contraction and the like.

It has now been found that the compounds defined in the preceding paragraphs are useful in the treatment of ocular complications. Further, among 13,14-dihydro-15-keto-PGs, (as either the carboxylic acid, salt, or ester) compounds having a 2,3-double bond, or a 5,6-triple bond, or compounds having substituents at any of C-3, C-6, C-16, C-17, C-19 and/or C-20 positions, or compounds having a lower alkyl or hydroxyalkyl group at the C-9 and/or C-11 position instead of the hydroxyl group, are particularly preferred compounds.

Further, it has been found that these 13,14-dihydro-15-keto-PGs are accompanied with no or extremely reduced peculiar central and peripheral physiological activities which are often caused by PGs, and further they have virtually no effects on enteron, trachea or bronchus which are characteristic for numerous PGs.

In the present invention, 13,14-dihydro-15-keto-PGs means PGs in which the carbon atoms at the 13-14 positions are saturated and carbon at the 15 position form a carbonyl group.

In this description, 13,14-dihydro-15-keto-PGs are named as follows, viz. the carbons constituting the α-chain, ω-chain and 5-membered ring are numbered according to the basic skeleton as follows:

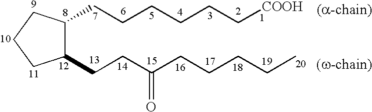

That is, in the basic skeleton, the constituent carbon atoms are numbered in such a way that the carboxylic acid carbon atom is C-1, the α-chain contain C-2-C-7, (the number increasing toward the ring), the five-membered ring contains C-8-C-12, and the ω-chain contains C-13-C-20. When there are fewer than 7 carbons of the α-chain, the numbers of the carbons following C-2 should be simply eliminated from 2 to 7 in this order, and when more than 7, the compound is named such that the “increase” is named as a substituent on the carbon at the 2 position. When the ω-chain contains fewer carbon atoms, they should be numbered correspondingly smaller than 20, and when more than 8, the carbon atoms at the 21 position and thereafter should be regarded as a substituent. As configuration, it is considered according to that of the above essential skeleton unless otherwise described.

For example, PGD, PGE and PGF mean compounds having a hydroxyl group at the C-9 and/or C-11 positions. In the present invention, PGs include compounds having a group other than a hydroxyl group on the C-9 and/or C-11 positions, such compounds being named as 9-dehydroxy-9-substituted or 11-dehydroxy-11-substituted compounds.

13,14-dihydro-15-keto-PGs used in the present invention may be 13,14-dihydro-15-keto-PG₁s containing a 5,6-single bond, 13,14-dihydro-15-keto-PG₂s containing a 5,6-double bond, 13,14-dihydro-15-keto-PG₃s containing both 5,6- and 17,18-double bonds may be used.

The typical examples of the 13,14-dihydro-15-keto-PGs used in the present invention are shown below:

13,13-dihydro-15-keto-PGA₁s, 13,14-dihydro-15-keto-PGA₂s,

13,14-dihydro-15-keto-PGA₃s, 13,14-dihydro-15-keto-PGB₁s,

13,14-dihydro-15-keto-PGB₂s, 13,14-dihydro-15-keto-PGB₃s,

13,14-dihydro-15-keto-PGC₁s, 13,14-dihydro-15-keto-PGC₂s,

13,14-dihydro-15-keto-PGC₃s, 13,14-dihydro-15-keto-PGD₁s,

13,14-dihydro-15-keto-PGD₂s, 13,14-dihydro-15-keto-PGD₃s,

13,14-dihydro-15-keto-PGE₁s, 13,14-dihydro-15-keto-PGE₂s,

13,14-dihydro-15-keto-PGE₃s, 13,14-dihydro-15-keto-PGF₁s,

13,14-dihydro-15-keto-PGF₂s, 13,14-dihydro-15-keto-PGF₃s,

13,14-dihydro-15-keto-PGJ₁s, 13,14-dihydro-15-keto-PGJ₂s,

13,14-dihydro-15-keto-PGJ₃s or the like.

These 13,14-dihydro-15-keto-PGs are particularly preferred compounds in treating ocular complications.

In the present invention, the addressed pharmacological effect is especially pronounced in FADs of the general formula (I):

[chemical structure]

(in which R is hydroxyl, hydroxyalkyl or alkyl);

Y is a saturated or unsaturated C₂₋₆ hydrocarbon chain (some of the carbon atoms constituting the hydrocarbon chain may form a carbonyl group, and the hydrocarbon chain may be substituted with one or more atoms or groups);

Z is a C₁₋₁₀ saturated or unsaturated hydrocarbon forming a straight-chain, branched-chain or ring (the hydrocarbon may be substituted with atoms or groups) or physiologically acceptable salts derived from the general formula [I] or those having an esterified carboxyl group.

A saturated or unsaturated C₂₋₆ hydrocarbon chain Y includes a straight hydrocarbon chain such as an alkyl, alkenyl, and alkynyl. A hydrocarbon chain with 6 carbons is preferred.

Examples of an unsaturated hydrocarbon chain include, for example, FADs in which the carbons at the 2-3 positions or 5-6 positions are joined by an unsaturated bond.

Some of the carbons forming the hydrocarbon chain Y may form a carbonyl group. A typical example includes 6-keto-FAD wherein the carbon at the 6 position constitutes a carbonyl group.

The hydrocarbon chain Y may be substituted with one or more atoms or groups. Such atoms or groups include, for example, a halogen atom such as a fluorine, chlorine or bromine atom; an alkyl group such as methyl, ethyl; a hydroxyl group. A typical example is one or more alkyl groups at the 3-carbon atom.

Z means a C₁₋₁₀ saturated or unsaturated hydrocarbon group. The hydrocarbon itself may form a ring or may be substituted with one or more atoms or groups.

As the hydrocarbon group Z, C₂₋₉ straight chain is particularly preferred. A hydrocarbon group with five carbons provides FADs with a ω-chain having eight carbons. accordingly, as described above, a hydrocarbon Z having more than 6 carbons is assumed to provide a substituent at the 20-carbon atom in the ω-chain (e.g. a hydrocarbon having seven carbons provides docohexanoic FADs).

The unsaturated bond (if present) may be at any position in Z. However, it is preferred that Z is unsaturated. Examples of the hydrocarbon Z forming a ring include a cyclo-pentyl or cyclohexyl group in which the carbons at 16 or 17 position in the ω-chain may be part of the ring.

Z may be substituted with one or more atoms or groups. Such atoms or groups include a halogen atom such as a fluorine, chlorine or bromine atom; an alkyl group such as a methyl, ethyl, isopropyl or isopropenyl group; an alkoxy group such as a methoxy or ethoxy group; a hydroxyl group; a phenyl group; and a phenoxy group. The position of the substituent atom(s) or group(s) is not limited, but typically, they may be at 16, 17, 19 and/or 20 position in the ω-chain.

Particularly preferred are compounds having one or two of the same or different atoms at the C-16 position, for example, a halogen atom such as a fluorine atom or a substituent, for example, an alkyl group such as a methyl, ethyl, hydroxy phenyl which may contain one or more substituents, benzyl, phenoxy, or cycloalkyl group such as a cyclopentyl or cyclohexyl group which contains the C-16 position as a constituent; compounds having an alkyl group such as methyl at the C-17 or C-19 position; and compounds having an alkyl group such as a methyl, ethyl, isopropyl, isopropenyl or alkoxy group such as a methoxy, ethoxy or propoxy group at the C-20 position. Highly preferred compounds carry a substituent at the C-17 position, said substituent being selected from cycloalkyl such as a cyclopentyl or cyclohexyl and phenyl being optionally substituted by hydroxy, halide such as chloride, bromide or fluoride. Strongly preferred is phenyl at C-17.

FADs may include the compounds of formula (I) which contain a hydroxyl group at the C-9 and/or C-11 position. In the present invention, FADs further include the compounds having a hydroxyalkyl or alkyl group instead of the hydroxyl group at the C-9 and/or C-11 position. Accordingly, the 13,14-dihydro-15-keto-FADs of the present invention include the compound of the general formula [I], wherein R is a hydroxyl, hydroxyalkyl or alkyl group. Such a hydroxyalkyl group is preferably a hydroxymethyl or 1-hydroxyethyl, 2-hydroxyethyl or 1-methyl-1-hydroxyethyl group. As the alkyl group, a lower alkyl group, especially a methyl or ethyl group is preferred.

The configuration of R for the carbon at the 9 and/or 11 position may be α, β or mixture thereof.

FADs of the present invention may be salts or esters. Such salts include physiologically acceptable salts, for example, those of an alkali metal such as sodium, potassium; those of an alkaline earth metal such as calcium, magnesium; those of an ammonium salt such as ammonia, methylamine dimethylamine, cyclopentylamine, benzylamine, piperidine, monoethanolamine, diethanolamine, monomethylmonoethanolamine, tromethamine, lysine, and tetralkylammonium salt. Such an ester includes, for example, methyl, ethyl, propyl, butyl, isopopyl, t-butyl, 2-ethylhexyl, straight or branched-chain alkyl ester which may contain an unsaturated bond; for example, ester having an alicyclic group such as a cyclopropyl, cyclopentyl or cyclohexyl group; an ester containing an aromatic group such as a benzyl or phenyl group (wherein the aromatic group may contain one or more substituents); a hydroxyalkyl or alkoxyalkyl ester such as a hydroxyethyl, hydroxyisopropyl, polyhydroxyisopropyl, methoxyethyl, ethoxyethyl or methoxyisopropyl group; an alkylsilyl ester e.g., a trimethylsilyl or triethylsilyl ester; a tetrahydropyranyl ester.

Preferred esters include, for example, a straight or branched lower alkyl ester such as a methyl, ethyl, propyl, n-butyl, isopropyl or t-butyl ester; or a benzyl ester; a hydroxyalkyl ester such as a hydroxyethyl or hydroxyisopropyl ester.

The carboxyl group at the C-1 position of 13,14-dihydro-15-keto-FADs of the present invention may be any of the above-described groups, Among them, esters, especially the C₁₋₁₄ alkyl ester are preferred.

13,14-Dihydro-15-keto-FADs of the present invention may include the isomers of the above compounds. Examples of such isomers include keto-hemiacetal tautomers between the C₆-carbonyl and C₉-hydroxyl, or the C₁₁-hydroxyl and C₁₅-carbonyl; optical isomers; and geometrical isomers.

Keto-hemiacetal tautomers between the C₁₁-hydroxyl group and C₁₅-carbonyl may be readily formed especially in 13,14-dihydro-15-keto-PGEs having an electrophilic group such as a fluorine atom at the C-16 position.

A mixture of the isomers, for example, a racemic mixture, tautomers of hydroxyl compound and hemiacetals may show similar effect as that shown by the respective compound.

In the present invention, especially preferred 13,14-dihydro-15-keto-FADs contain a 5,6-single or double bond, or a carbonyl group at the C-6 position. Other preferred compounds are 13,14-dihydro-15-keto-FADs having 20-24 carbon atoms. Still other preferred compounds are 13,14-dihydro-15-keto-FADs wherein the carbon atom at the 16 position is substituted with a halogen atom or an alkyl group. Further preferred compounds are 13,14-dihydro-15-keto-FADs having more than 20 carbons and an alkyl group at C-19 position.

Particularly, the compounds having a C₁₋₄ alkyl, (for example, a methyl, ethyl, propyl or butyl group) at the C-20 position (i.e. having a prolonged ω-chain) are preferred.

That is, in 13,14-dihydro-15-keto-FADs used in the present invention, those having an alkyl group at the C-20 position provide a particularly beneficial result, irrespective of the structure of the five-membered ring, or the existence of double bond or other substituents. Particularly, those wherein the alkyl group is ethyl (wherein the ω-chain contains a C₁₀ straight chain) show pronounced pharmacological effect.

The above 13,14-dihydro-15-keto-FADs of the present invention may be prepared according to the methods described, for example, in Japanese Patent Application Nos. 63-18326, 63-18327 and 63-108329. These disclosures are to be considered to be incorporated in the present description.

1. Method for the treatment of ocular complications selected from the group of vasospastic glaucoma, vasospastic syndrome, vascular dysregulation syndrome, and signs of vascular dysregulation, especially in patients presenting risk factors for glaucoma, such as but not exclusively high intraocular pressure, family history of glaucoma, glaucoma in the contralateral eye and high myopia, comprising in particular the topical, preferably the topical ocular administration of a fatty acid derivative (FAD) to an individual in need of such treatment.

2. Method of claim 1, wherein said FAD is 13,14-dihydro-15-keto-FAD in accordance to formula (I), [chemical structure]
(in which R is hydroxyl, hydroxyalkyl or alkyl);
Y is a saturated or unsaturated C₂₋₆ hydrocarbon chain (some of the carbon atoms constituting the hydrocarbon chain may form a carbonyl group, and the hydrocarbon chain may be substituted with one or more atoms or groups);
Z is a C₁₋₁₀ saturated or unsaturated hydrocarbon forming a straight-chain, branched-chain or ring (the hydrocarbon may be substituted with atoms or groups) or physiologically acceptable salts derived from the general formula [I] or those having an esterified carboxyl group.

3. Method of claim 2, wherein the carboxyl group at the end of the α-chain is in the form of an alkyl ester.

4. Method of claim 2, wherein an alkyl group is attached at the C-20 position.

5. Method of claim 2, wherein said FAD is a 13,14-dihydro-15-keto-17-phenyl, 18, 19, 20-trinor prostaglandin.

6. Method of claim 2, wherein said FAD is 13,14-dihydro-15-keto-docohexanoic acid isopropyl ester.
